(19) United States
(12) Patent Application Publication
(10) Pub. No.: US 2024/0143729 A1
(43) Pub. Date: May 2, 2024

(54) MEDICAL IMAGING DEVICE AND METHOD FOR PROVIDING A USER INTERFACE FOR A MEDICAL ACCESSORY DEVICE

(71) Applicant: Siemens Healthcare GmbH, Erlangen (DE)

(72) Inventors: Yufan ZHAO, Erlangen (DE); Ralf GUTJAHR, Nuernberg (DE); Markus URBANSKI, Forchheim (DE)

(73) Assignee: Siemens Healthcare GmbH, Erlangen (DE)

(21) Appl. No.: 18/499,752

(22) Filed: Nov. 1, 2023

(30) Foreign Application Priority Data
Nov. 2, 2022 (EP) 22205134.4

(51) Int. Cl. G06F 21/44; G06F 13/42

(52) U.S. Cl. CPC G06F 21/44; G06F 13/4282; G06F 2213/0002

(57) ABSTRACT

A medical imaging device, comprises: a first communication interface configured to transfer authentication data; and a second communication interface configured to transfer user interface data; wherein the medical imaging device is configured to provide a user interface for a medical accessory device based on the authentication data and the user interface data.

### CROSS-REFERENCE TO RELATED APPLICATION(S)

The present application claims priority under 35 U.S.C. § 119 to European Patent Application No. 22205134.4, filed Nov. 2, 2022, the entire contents of which is incorporated herein by reference.

### FIELD

In one aspect, one or more example embodiments of the present invention relate to a medical imaging device. In another aspect, one or more example embodiments of the present invention relate to a method for providing a user interface for a medical accessory device.

### BACKGROUND

Integration of medical devices into a clinical digital ecosystem, in particular with regard to remote control and assistance, is becoming increasingly important.

WO 2022/015560 A1 discloses a system for enabling communication between a fluid injection system and at least one of a plurality of external systems.

### SUMMARY

An underlying technical problem of embodiments of the present invention is to facilitate an integration of a medical accessory device into a medical imaging workflow. At least this problem is solved by the subject matter of the independent claims and the disclosure. The dependent claims are related to further aspects of embodiments of the present invention.

Independent of the grammatical term usage, individuals with male, female or other gender identities are included within the term.

An embodiment of the present invention relates in one aspect to a medical imaging device, comprising:
a first communication interface for transferring authentication data,
a second communication interface for transferring user interface data,
the medical imaging device being configured for providing a user interface for a medical accessory device based on the authentication data and the user interface data.

The first communication interface may be a serial bus communication interface, in particular a linear serial bus communication interface. The first communication interface may be configured for multi-master and/or deterministic network communication, in particular via a wired one-to-one physical connection.

The first communication interface may be a controller area network interface, in particular according to a CAN bus protocol.

The second communication interface may be a switched network interface. The second communication interface may be configured for non-deterministic network communication.

The second communication interface may be an Ethernet interface, in particular according to a Local Area Network protocol.

The user interface for the medical accessory device may be a graphical user interface for the medical accessory device. The user interface data may comprise data entered into the user interface and/or data to be output via the user interface. The user interface data may comprise data provided by the medical accessory device and/or data intended for the medical accessory device. The user interface data may be transferred through the second communication interface by receiving, through the second communication interface, the data provided by the medical accessory device and/or by sending, through the second communication interface, the data intended for the medical accessory device.

The data intended for the medical accessory device may comprise patient data, in particular patient data needed for a patient-specific protocol adjustment by the medical accessory device, and/or scan data, in particular scan data needed for a scan-specific protocol adjustment by the medical accessory device. The patient data may comprise, for example, patient identity information, patient age, patient sex, patient weight and/or patient height. The scan data may comprise, for example, kV-data, scan protocol data, scan timestamp data and/or test bolus data.

The second communication interface may be different from the first communication interface, in particular in terms of communication network and/or in terms of communication protocol.

The authentication data may comprise a public key of the medical imaging device and/or a public key of the medical accessory device. In particular, the authentication data comprising the public key of the medical imaging device may be sent through the first communication interface to the medical accessory device. In particular, the authentication data comprising the public key of the medical accessory device may be sent through the first communication interface to the imaging accessory device.

According to one example, the medical accessory device is configured as a server for the communication between the medical imaging device and the medical accessory device through the second communication interface and the medical imaging device is configured as a client for the communication between the medical imaging device and the medical accessory device through the second communication interface. In particular, the medical imaging device may be configured simultaneously as a client for the communication between the medical imaging device and the medical accessory device through the second communication interface and as a server for the communication between the medical imaging device and the remote console through the second communication interface. The remote console may be configured as a client for the communication between the medical imaging device and the remote console through the second communication interface.

According to another example, the medical accessory device is configured as a client for the communication between the medical imaging device and the medical accessory device through the second communication interface and the medical imaging device is configured as a server for the communication between the medical imaging device and the medical accessory device through the second communication interface.

For the communication between the medical imaging device and the medical accessory device through the second communication interface, a HTTPS communication protocol, in particular a TLS-based HTTPS communication protocol, may be used to ensure secure communication. A public key certificate self-signed by the server may be provided by the server to the client, for example, by the medical accessory device to the medical imaging device, and/or may be trusted by the client. In particular, the public key certificate self-signed by the server may be received by the client through the second communication interface.

A public key of the client may be sent by the client to the server through the first communication interface, in particular to enable the server to authenticate the client. An access token, for example, in form of a random number, may be generated by the server. The access token may be generated by the server specifically for a current communication session and/or may be valid until the end of the current communication session. The access token may be encrypted by the server based on the public key of the client. The encrypted access token may be received by the client through the second communication interface and decrypted by the client with the corresponding private key of the client.

The access token may be then attached by the client to a dataset, in particular to a header of a massage of the dataset. The dataset may be sent to the server through the second communication interface. The dataset may comprise an instruction from the client for the server. The server may be configured to check the attached access token and to follow the instruction from the client only if the attached access token matches the access token, that has been originally generated by the server. This allows the server to ensure, that the dataset has been sent by the same client as the one that has sent the public key through the first communication interface, for example via a first communication line that connects the client one-to-one to the server.

The medical accessory device may send a byte stream or an address of stream to the second communication interface. The medical imaging device may receive a URL of the user interface from the server through the second communication interface and/or acquire the user interface in form of a URL or bytes (bit-stream) into the imaging control system.

For the communication between the medical imaging device and the remote console through the second communication interface, a HTTPS communication protocol, in particular a TLS-based HTTPS communication protocol, may be used to ensure secure communication, for example, in form of a virtual private network (VPN). The remote console may be configured to verify an authentication token provided by the medical imaging device through the second communication interface.

The medical accessory device may be selected from the group consisting of a fluid injection device, a biosignal measuring device, a positioning device, a medical treatment device and combinations thereof. The fluid injection device may be, for example, a contrast agent injection device. The biosignal measuring device may be, for example, an electrocardiogram device. The positioning device may be configured, for example, for patient positioning and/or for interventional apparatus positioning, in particular for needle positioning and/or for catheter positioning. The medical treatment device may be configured, for example, for radiation therapy and/or for robotic surgery

If the medical accessory device is a fluid injection device, the data provided by the medical accessory device may comprise injection protocol data. If the medical accessory device is a biosignal measuring device, in particular an electrocardiogram device, the data provided by the medical accessory device may comprise biosignal data, in particular electrocardiogram data. If the medical accessory device is a positioning device, for example, for patient positioning and/or for interventional apparatus positioning, the data provided by the medical accessory device may comprise position data. If the medical accessory device is a medical treatment device, in particular for radiation therapy and/or for robotic surgery, the data provided by the medical accessory device may comprise treatment protocol data.

In another aspect, the medical imaging device is selected from the group consisting of a computed tomography device, a magnetic resonance imaging device, an ultrasound imaging device and a projection X-ray imaging device. In particular, the medical imaging device may be a computed tomography device. The projection X-ray imaging device may be, for example, an angiography imaging device and/or a fluoroscopy imaging device.

An embodiment of the present invention relates in one aspect to a medical imaging system, comprising the medical imaging device according to one of the aspects of embodiments of the present invention and a remote console for the medical imaging device, the medical imaging system being configured for relaying the user interface for the medical accessory device from the medical imaging device to the remote console, the remote console being configured for providing the user interface for the medical accessory device. The medical imaging system may further comprise the medical accessory device. The medical imaging system may comprise a serial bus, to which the first communication interface is connected, and/or a switched network, to which the second communication interface is connected.

This allows remote operation of the medical accessory device using the remote console for the medical imaging device, thereby making the remote session more efficient. In particular, this allows a remote user to select injection protocols from the injector's library/repository during a remote scan examination. Injection parameters can be optimized patient-specific on the remote console by the remote user.

Between the medical imaging device and the medical accessory device, a first dataset may be exchanged through the first communication interface and a second dataset may be exchanged through the second communication interface. Between the medical imaging device and the remote console, the first dataset and the second dataset both may be exchanged through the second communication interface. The first dataset may comprise a synchronous start signal, a synchronous stop signal, protocol synchronization information and/or operational report information. The second dataset may comprise the user interface data.

An embodiment of the present invention relates in one aspect to a method for providing a user interface for a medical accessory device, the method comprising:
Transferring authentication data through a first communication interface of a medical imaging device,
Transferring user interface data through a second communication interface of the medical imaging device,
Providing, by the medical imaging device, the user interface for the medical accessory device based on the authentication data and the user interface data.

The method may further comprise:
Relaying the user interface for the medical accessory device from the medical imaging device to a remote console, in particular through the second interface of the medical imaging device,
Providing, by the remote console, the user interface for the medical accessory device.

The user interface for the medical accessory device may be relayed from the medical imaging device to a remote console by encrypting the user interface by the medical imaging device, sending the encrypted user interface through the second communication interface from the medical imaging device to a remote console and decrypting the encrypted user interface by the remote console.

Data, in particular the authentication data and the user interface data, can be received, for example, by receiving a signal that carries the data and/or by reading the data from a computer memory and/or by a manual user input, for example, through a graphical user interface. The user interface may be provided to a user, for example, by a human-machine-interface, in particular by activating an output layer of the user interface, in particular to be perceived by the user, and by activating an input layer of the user interface, in particular to be ready to receive input from the user. The human-machine-interface may comprise, for example, input hardware such as a keyboard, a computer mouse or a touchsensitive surface and output hardware such as a computer screen, a loudspeaker and a printer. The human-machine-interface may be, for example, a touchsensitive screen.

In the context of the present invention, the expression “based on” can in particular be understood as meaning “using, inter alia”. In particular, wording according to which a first feature is calculated (or generated, determined etc.) based on a second feature does not preclude the possibility of the first feature being calculated (or generated, determined etc.) based on a third feature.

Reference is made to the fact that the described methods and the described systems are merely preferred example embodiments of the present invention, and that the present invention can be varied by a person skilled in the art, without departing from the scope of the present invention as it is specified by the claims.

### BRIEF DESCRIPTION OF THE DRAWINGS

The present invention will be illustrated below with reference to the accompanying figures using example embodiments. The illustration in the figures is schematic and highly simplified and not necessarily to scale.

FIG. 1 shows a medical imaging device.

FIG. 2 shows a medical imaging system.

FIG. 3 shows a flow chart for a method for providing a user interface for a medical accessory device.

FIG. 4 shows a flow chart for the method for providing the user interface for the medical accessory device by a remote console.

### DETAILED DESCRIPTION

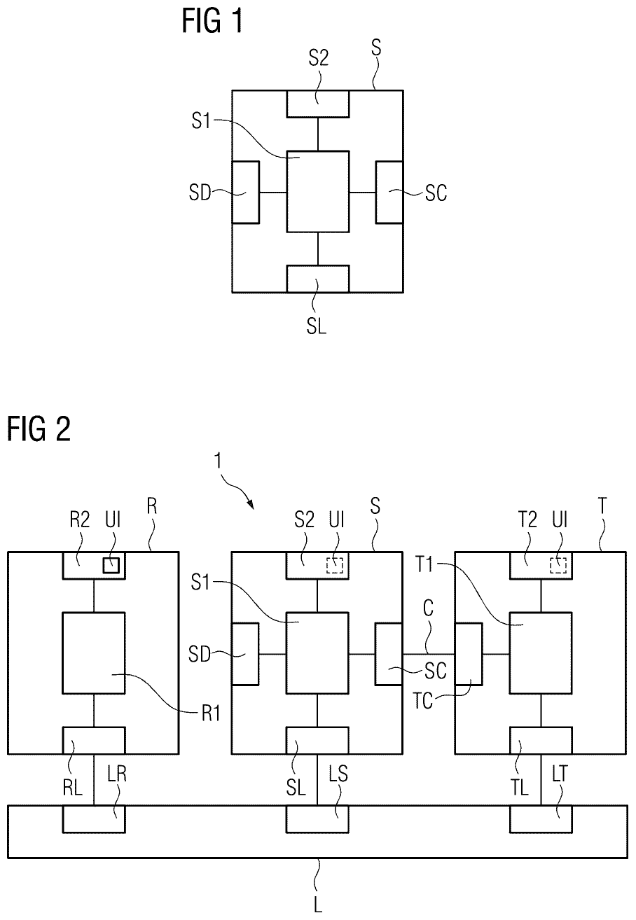

FIG. 1 shows the medical imaging device S, comprising a first communication interface SC for transferring M1 authentication data and a second communication interface SL for transferring M2 user interface data. The medical imaging device S is configured for providing M3 a user interface UI for a medical accessory device T based on the authentication data and the user interface data. The first communication interface SC is a serial bus communication interface in form of a controller area network interface. The second communication interface SL is a switched network interface in form of an Ethernet interface.

The medical imaging device S comprises the imaging data acquisition system SD for generating medical imaging data by executing a scan of an examination object based on a scan protocol. The medical imaging device S comprises the data processing system S1 for processing the authentication data, the user interface data and the medical imaging data. The data processing system S1 may be the imaging control system of the medical imaging device. The medical imaging device S comprises the human-machine-interface S2 for providing the user interface UI to a user at the location of the medical imaging device S. The human-machine-interface S2 is further configured to provide a user interface for the medical imaging device S to a user at the location of the human-machine-interface S2 of the medical imaging device S. The human-machine-interface S2 of the medical imaging device S may be located, for example, in a control room. The data acquisition system SD may be located, for example, in a scan room.

FIG. 2 shows the medical imaging system 1, comprising the medical imaging device S, the remote console R for the medical imaging device S and further comprising the medical accessory device T. The medical imaging system 1 comprises the switched network L and the wired physical one-to-one connection C and is configured for relaying M4 the user interface UI for the medical accessory device T from the medical imaging device S to the remote console R through the second communication interface SL, the switched network L and the remote console interface RL.

The remote console R is configured for providing M5 the user interface UI for the medical accessory device T. The remote console R comprises the data processing system R1 for processing the relayed user interface UI. The remote console R comprises the human-machine-interface R2 for providing the user interface UI to a user at the location of the remote console R. The human-machine-interface R2 is further configured to provide a user interface for the medical imaging device S to a user at the location of the human-machine-interface R2 of the remote console R.

The medical accessory device T is a fluid injection device, a biosignal measuring device, a positioning device or a medical treatment device. The medical accessory device T comprises the first communication interface TC of the medical accessory device T for transferring the authentication data and the second communication interface TL of the medical accessory device T for transferring the user interface data. The medical accessory device T comprises the data processing system T1 for processing the authentication data and the user interface data. The medical accessory device T comprises the human-machine-interface T2 for providing the user interface UI to a user at the location of the human-machine-interface T2 of medical accessory device T. The human-machine-interface T2 of the medical accessory device T may be located, for example, in a control room and/or in a scan room. The medical accessory device T may comprise a subsystem, the subsystem being located in the scan room and being connected to the human-machine-interface T2 that is located in the control room. Such a subsystem may comprise, for example, an injector head, a biosignal sensor, patient positioning mechanics and/or a robotic arm.

The first communication interface SC of the medical imaging device S is connected to the first communication interface TC of the medical accessory device T via a serial bus in form of the wired physical one-to-one connection C. The second communication interface SL of the medical imaging device S is connected to the port LS of the switched network L. The second communication interface TL of the medical accessory device T is connected to the port LT of the switched network L. The remote console interface RL is connected to the port LR of the switched network L.

In addition to the medical imaging device S and the medical accessory device T, a further medical imaging device and a further medical accessory device may be connected to the switched network L and thereby to the remote console R. The further medical imaging device and the further medical accessory device may be connected to each other via another wired physical one-to-one connection for transferring further authentication data. A user interface for the further medical accessory device may be provided by the further medical imaging device and relayed from the further medical imaging device to the remote console R as described above for the medical imaging device S and the medical accessory device T.

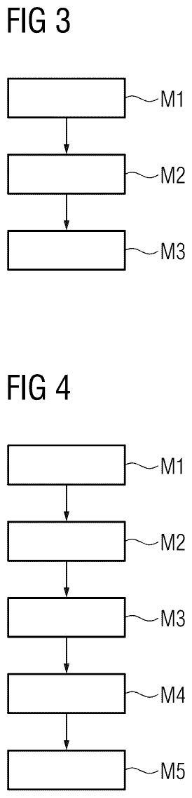

FIG. 3 shows a flow chart for a method for providing the user interface UI for the medical accessory device T, the method comprising:
Transferring M1 authentication data through the first communication interface SC of the medical imaging device S,
Transferring M2 user interface data through the second communication interface SL of the medical imaging device S,
Providing M3, by the medical imaging device S, the user interface UI for the medical accessory device T based on the authentication data and the user interface data.

FIG. 4 shows a flow chart for the method for providing the user interface UI for the medical accessory device T by the remote console R, the method further comprising:
Relaying M4 the user interface UI for the medical accessory device T from the medical imaging device S to a remote console R,
Providing M5, by the remote console R, the user interface UI for the medical accessory device T.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A medical imaging device, comprising:
a first communication interface configured to transfer authentication data; and
a second communication interface configured to transfer user interface data; wherein
the medical imaging device is configured to provide a user interface for a medical accessory device based on the authentication data and the user interface data.

2. The medical imaging device according to claim 1, wherein the first communication interface is a serial bus communication interface.

3. The medical imaging device according to claim 1, wherein the first communication interface is a controller area network interface.

4. The medical imaging device according to claim 1, wherein the second communication interface is a switched network interface.

5. The medical imaging device according to claim 1, wherein the second communication interface is an Ethernet interface.

6. The medical imaging device according to claim 1, wherein the medical accessory device is a fluid injection device, a biosignal measuring device, a positioning device, a medical treatment device, or a combination thereof.

7. The medical imaging device according to claim 1, wherein the medical imaging device is a computed tomography device, a magnetic resonance imaging device, an ultrasound imaging device or a projection X-ray imaging device.

8. A medical imaging system, comprising:
the medical imaging device according to claim 1; and
a remote console for the medical imaging device; wherein
the medical imaging system is configured to relay the user interface for the medical accessory device from the medical imaging device to the remote console, and
the remote console is configured to provide the user interface for the medical accessory device.

9. A method for providing a user interface for a medical accessory device, the method comprising:
transferring authentication data via a first communication interface of a medical imaging device;
transferring user interface data via a second communication interface of the medical imaging device; and
providing, by the medical imaging device, the user interface for the medical accessory device based on the authentication data and the user interface data.

10. The method according to claim 9, wherein the first communication interface is a serial bus communication interface.

11. The method according to claim 9, wherein the first communication interface is a controller area network interface.

12. The method according to claim 9, wherein the second communication interface is a switched network interface.

13. The method according to claim 9, wherein the second communication interface is an Ethernet interface.

14. The method according to claim 9, wherein the medical accessory device is a fluid injection device, a biosignal measuring device, a positioning device, a medical treatment device or a combination thereof.

15. The method according to claim 9, further comprising:
relaying the user interface for the medical accessory device from the medical imaging device to a remote console; and
providing, by the remote console, the user interface for the medical accessory device.

16. The medical imaging device according to claim 2, wherein the second communication interface is a switched network interface.

17. The medical imaging device according to claim 3, wherein the second communication interface is a switched network interface.

18. The medical imaging device according to claim 2, wherein the second communication interface is an Ethernet interface.

19. The medical imaging device according to claim 3, wherein the second communication interface is an Ethernet interface.

20. A medical imaging system, comprising:
the medical imaging device according to claim 1; and
a remote console for the medical imaging device; wherein
the medical imaging system is configured to relay the user interface for the medical accessory device from the medical imaging device to the remote console,
the remote console is configured to provide the user interface for the medical accessory device,
the first communication interface is a serial bus communication interface or a controller area network interface, and
the second communication interface is a switched network interface or an Ethernet interface.
